(19) United States
(12) Patent Application Publication
(10) Pub. No.: US 2021/0169586 A1
(43) Pub. Date: Jun. 10, 2021

(54) SYSTEM AND METHOD FOR GUIDED PLACEMENT OF MEDICAL INSTRUMENT

(71) Applicant: Xoran Technologies LLC, Ann Arbor, MI (US)

(72) Inventors: William C. VAN KAMPEN, Saline, MI (US); Miodrag RAKIC, Saline, MI (US)

(21) Appl. No.: 17/114,357

(22) Filed: Dec. 7, 2020

Related U.S. Application Data
(60) Provisional application No. 62/945,100, filed on Dec. 6, 2019.

(51) Int. Cl. A61B 34/20; A61B 34/00; A61B 34/10; A61B 90/00

(52) U.S. Cl. CPC A61B 34/20; A61B 34/25; A61B 34/10; A61B 2034/107; A61B 90/37; A61B 2090/3762; A61B 2090/3966; A61B 90/39

(57) ABSTRACT

A system and method for a guided placement of a medical instrument n a patient using a computed topography (CT) scanner includes an instrument placement kit having an alignment frame and multiple guidance devices, and a user interface unit. The alignment frame is configured to be attached to a body of the patient and also be used as a reference plane in the scanned images, and the multiple guidance devices are configured for selective engagement with the alignment frame and also have different sets of pre-defined parameters. The user interface unit is operable to determine a planned trajectory based on the scanned images and also determine the prescribed parameters among the different sets of pre-defined parameters such that the user interface unit allows a user to select one of the guidance devices according to the prescribed parameters for guiding the medical instrument along the planned trajectory.

### RELATED APPLICATION

This application claims the benefit of U.S. Provisional Application No. 62/945,100, filed Dec. 6, 2019, which is incorporated herein by reference in its entirety.

### FIELD

The present disclosure relates to a medical instrument placement, and more particularly to a system and method for a trajectory guidance placement of the medical instrument.

### BACKGROUND

The statements in this section merely provide background information related to the present disclosure and may not constitute prior art.

Medical procedures involving precision insertion and placement of a therapy device into a patient through a body portal are used to treat a variety of medical condition. For example, External Ventricular Drains (EVDs) are frequently placed by hand, which leads to a significant rate of complications such as a surface vein violation because missing the ventricles can lead to improper drain function or necessitate reinsertion, reinserting catheter or other medical instrument multiple times can needlessly damage healthy brain tissue, and inserting of catheter or other medical instruments may violate surface veins, which can lead to intracranial bleeding and/or other complications.

To accurately place the medical instrument, surgeons typically use conventional surgical navigation systems and methods such as frame or frameless stereotactic apparatus procedures. The stereotactic apparatus of varying configurations are well known device. For example, a “center-of-arc” stereotactic apparatus includes an arc-shaped frame and a pivot about which the frame is movable, and aligns with the target location. As a result, multiple device trajectory and entry points are available to reach the target location. Other stereotactic systems may utilize what is referred to as “frameless” or “microframe” technology. These systems typically utilize a pre-aligned, stereotactic platform custom-made for a particular patient's cranial physiology. Such systems may allow pre-operative alignment and configuration (potentially reducing the patient's time in the operating room) and may further result in less discomfort to the patient.

However, we have discovered that while providing accurate device placement with the microframe systems, the custom-made platform presents a recurring fee for each patient as compared to re-useable platform. Moreover, it may take days to receive the custom platform after an order is placed, reducing opportunity to offer same-day planning and surgery. Still further, such custom-made systems may have little or no ability to accommodate subsequent targeting adjustments when needed (for example, when a large blood vessel is later found within the planned insert trajectory). Accordingly, the conventional stereotactic procedures are time-consuming and require costly capital equipment.

### SUMMARY

The present disclosure relates to a system and method for a guided placement of a medical instrument in a patient using a computed tomography (CT) scanner providing scanned images of the patient including a target site for the medical instrument. The guided placement system of the present disclosure improves accuracy over the hand placement of the medical instrument, and have benefits for ease of use, simplified procedure, and reduced operating time. In addition, the guidance placement system may have a disposable instrument placement kit option for using a single time to guide the placement of the medical instrument.

According to an aspect of the present disclosure, the guidance placement system includes an instrument placement kit (device) having an alignment frame configured to be attached to a body of the patient and one or more guidance devices each configured for selective engagement with the alignment frame. The alignment frame has one or more identifiers to define a reference plane that can be determined from scanned images from the CT scanner. Each guidance device has a set of pre-defined parameters and also the multiple guidance devices each has different sets of the pre-defined parameters for guiding the placement of the medical instrument. The guidance placement system further includes a user interface unit operable to determine the pre-defined parameters of the multiple guidance devices from the scanned images including the reference plane of the alignment frame. In addition, the user interface unit is operable to compute a planned trajectory of the medical instrument based on the target site and the reference plane of the alignment frame, the user interface unit is further operable to determine prescribed parameters among the different sets of pre-defined parameters and select a guidance device from the multiple guidance devices, and the user interface unit is operable to indicate the selected guidance and prescribed parameters such that the selected guidance device can be selectively mounted to the alignment frame according to the prescribed parameters to guide the medical instrument along the planned trajectory.

According to a further aspect of the present disclosure, the alignment frame is formed as a ring shape with markings around a perimeter of the alignment frame for indicating a rotational position of the selected guidance device when the guidance device is engaged with the alignment frame.

According to a further aspect of the present disclosure, each of the guidance devices is formed as a circular shape having an upper flange and a bottom disc extending from a plane of the upper flange such that each of the guidance devices is formed with the upper flange and the bottom disc, which are parallel and connected with a circular side wall. The upper flange of the guidance device has a marker to align with one of markings formed around a perimeter of the alignment frame to indicate an insertion direction of the medical instrument as one of the pre-defined parameters. The bottom disc of the guidance device is formed with multiple holes arranged as a honeycomb pattern for indicating an insertion location of the medical instrument as one of the pre-defined parameters, and all of the holes formed through the bottom disc of the guidance device are parallel to each other and are at a same angle, which is inclined relative to a top surface of the bottom disc for indicating an insertion angle of the medical instrument as one of the pre-defined parameters.

According to a further aspect of the present disclosure, the instrument kit further includes oen or more sleeves each having different pre-defined lengths for guiding the placement of the medical instrument such that the user interface unit is operable to determine a prescribed length among the different pre-defined lengths based on the planned trajectory. The user interface unit is further operable to select a sleeve from the multiple sleeves based on the prescribed length such that the selected sleeve can be selectively engaged with the selected guidance device mounted to the alignment frame. In addition, the selected sleeve is inserted into one of multiple holes formed through a bottom disc of the selected guidance device determined as an entry location of the medical instrument, and each of the sleeves has a chamfered end for engaging with an entry hole drilled on the body of the patient for stability when the medical instrument is placed in the body of the patient along the planned trajectory.

According to another aspect of the present disclosure, each of the guidance devices is formed as a circular shape having an outer ring and a middle rail formed along a center line of the outer ring such that each of the guidance devices is formed with the outer ring and the middle rail in a single plane. The outer ring of the guidance device is formed with a marker to align with one of markings formed around a perimeter of the alignment frame and the middle rail of the guidance device is formed with multiple holes arranged in a line such that the marker and holes of the guidance device mounted to the alignment frame indicate an insertion location of the medical instrument as one of the pre-defined parameters.

According to a further aspect of the present disclosure, the instrument placement kit further includes multiple guide carriages each having different sets of pre-defined parameters such as an insertion angle and an insertion direction of the medical instrument for guiding the placement of the medical instrument such that the user interface unit is operable to select one among the multiple guide carriages determined from the prescribed parameters based on the planned trajectory.

According to another aspect of the present disclosure, a method for a guided placement of a medical instrument in a patient includes the steps of providing an instrument placement kit, placing an alignment frame onto a body of the patient adjacent a determined entry point, acquiring images including the alignment frame scanned by a computed tomography (CT) scanner, determining a planned trajectory relative to an anatomy of the patient and the alignment frame, determining prescribed parameters from a set of pre-defined parameters and indicating to an operator the prescribed parameters, mounting one of multiple guidance devices selectively to the alignment frame according to the prescribed parameters, and positioning the medical instrument along the guidance devices mounted to the alignment frame such that the medical instrument is placed along the planned trajectory.

According to a further aspect of the present disclosure, the step of determining the planned trajectory includes the step of indicating a target site in the acquired scanned images having the anatomy of the patient and a reference plane identified as the alignment frame.

According to a further aspect of the present disclosure, the step of mounting one of the multiple guidance devices selectively to the alignment frame includes the steps of selecting one of the guidance devices each having one inclined angle of multiple holes formed in each of the guidance devices according to a prescribed hole angle, and rotatably positioning the selected guidance device in the alignment frame according to a prescribed ring angle.

According to a further aspect of the present disclosure, the step of positioning the medical instrument along the guidance devices mounted to the alignment frame includes the steps of selecting one of multiple holes formed in each of the guidance devices as an insertion location of the medical instrument according to a prescribed hole number, and making an entry hole on the body of the patient at the insertion location of the medical instrument through the selected hole of the selected guidance device engaged with the alignment frame.

According to a further aspect of the present disclosure, the method further includes the step of providing multiple sleeves each having different pre-defined lengths for guiding an insertion length of the medical instrument through the entry hole on the body of the patient. The step of providing the multiple sleeves includes the steps of selecting one sleeve among the multiple sleeves having the different pre-defined lengths according to a prescribed length determined from the planned trajectory, inserting the selected sleeve into the selected hole of the selected guidance device, and engaging a chamfered end formed in each of the sleeves with the entry hole on the body of the patient.

Further details and benefits will become apparent from the following detailed description of the appended drawings. The drawings are provided herewith purely for illustrative purposes and are not intended to limit the scope of the present disclosure.

### BRIEF DESCRIPTION OF THE DRAWINGS

In order that the disclosure may be well understood, there will now be described various forms thereof, given by way of example, reference being made to the accompanying drawings, in which:

FIG. 1 is a diagram of a guidance placement system in accordance with an exemplary form of the present disclosure;

FIG. 2 is a detailed perspective view of an instrument placement kit placed onto a body of a patient according to the guidance placement system of FIG. 1, FIG. 2A is a detailed perspective view of the assembly of the instrument placement kit of FIG. 2, and FIG. 2B is a side view of the assembly of the instrument placement kit of FIG. 2;

FIG. 3A is a top view of an alignment frame in the instrument placement kit of FIG. 2, FIG. 3B is a side view of the alignment frame in the instrument placement kit of FIG. 2, and FIG. 3C is a top view of the alignment frame having a locking feature in the instrument placement kit of FIG. 2;

FIGS. 4(A), 4(B), and 4(C) are top views of multiple guidance devices in the instrument placement kit of FIG. 2;

FIG. 5 is a side view of one of the multiple guidance devices in the instrument placement kit of FIG. 2;

FIGS. 6(A), 6(B), and 6(C) are isometric views of multiple sleeves in the instrument placement kit of FIG. 2;

FIG. 7 is a detailed top view of an assembly of an instrument placement kit in accordance with another exemplary form of the present disclosure, and FIGS. 7(A), 7(B), and 7(C) are side views of multiple guide carriages in the instrument placement kit of FIG. 7;

FIG. 8 shows a display screen showing scanned images and prescribed parameters as an output in a user interface unit of the guidance placement system of FIG. 1; and

FIG. 9 shows a flow diagram of a method for a guidance placement of a medical instrument in accordance with an exemplary form of the present disclosure.

The drawings described herein are for illustration purposes only and are not intended to limit the scope of the present disclosure in any way.

### DETAILED DESCRIPTION

The following description is merely exemplary in nature and is in no way intended to limit the present disclosure or its application or uses. It should be understood that throughout the drawings, corresponding reference numerals indicate like or corresponding parts and features.

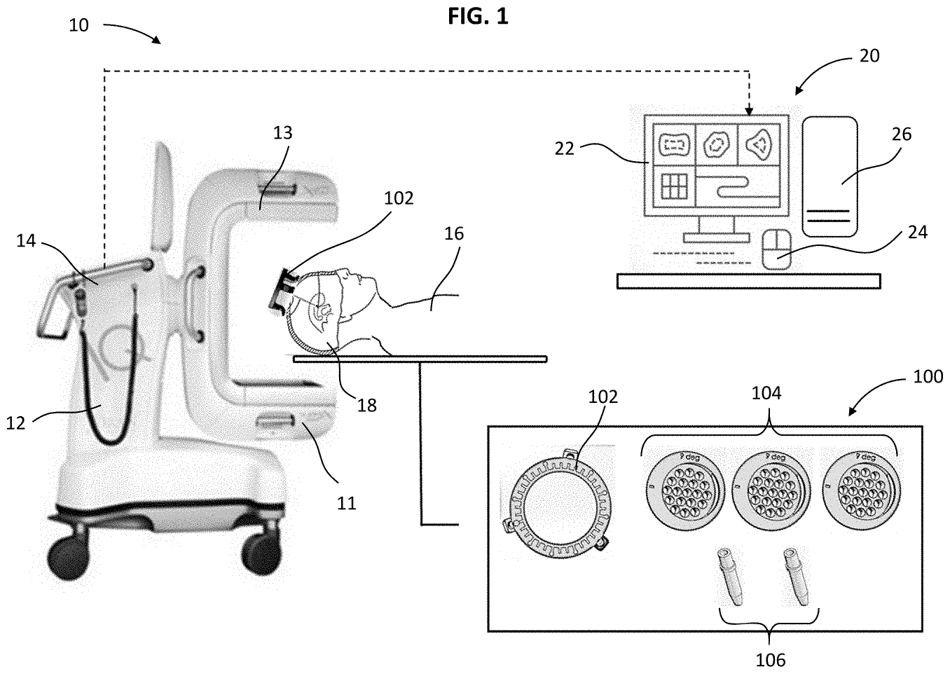

The present disclosure is directed toward a system and method for the guided placement of the medical instrument. In particular, the present disclosure relates to the system and method for image-guided placement of a surgical or cranial instrument in a body of the patient. The system and method of the present disclosure could also be used for a biopsy needle or other intracranial procedure, and the system can be used with various patients and in various regions of a patient's body, The system and method can use of an instrument placement kit 100 to guide the placement of the medical instrument. The instrument placement kit 100 including a computed tomography (CT) scanner 12 and a user interface unit 20 such as a computer system having a processor and a memory can be used in an operating room as illustrated in FIG. 1.

With reference to FIG. 1, the guidance placement system 10 that can be used for various procedures is illustrated. The guidance placement system 10 can be used to guide the placement of the External Ventricular Drain (EVD) catheter relative to a patient 16 to assist in the implementation or performance of a surgical procedure. It should be noted that the guidance placement system 10 may be used to guide other devices including: catheters, probes, needles, leads, implants, etc. According to various embodiments, examples include ablation catheters, deep brain simulation (DBS) or macro-electrodes or leads, micro-electrodes (ME) or leads for recording, etc. Moreover, the guided medical device may be used in any region of the body. The guidance placement system 10 and the various medical devices may be used in any appropriate procedure, such as one that is generally minimally invasive, arthroscopic, percutaneous, or an open procedure. Although an exemplary guidance placement system 10 including an imaging device 12 are discussed herein, one skilled in the art will understand that the disclosure is merely for clarity of the present discussion and any appropriate imaging system, guidance system, patient specific data, and non-patient specific data can be used. For example, the interoperative imaging system can include a computed tomography (CT) scanner such as a XCAT™IQ sold by Xoran Technologies, LLC., and also disclosed in U.S. Patent Publication No. U.S. Pat. No. 9,055,874 B2, filed on Jan. 28, 2008, etitled “Motion tracker to detect and correct for movement of a patient in a CT scanner”, incorporated herein by reference. In addition, another image system disclosed in U.S. Patent Publication No. U.S. Pat. No. 8,303,181 B2, filed on Aug. 9, 2004, etitled “Intraoperative collapsable CT imaging system”, incorporated herein by reference, can be used. It will be understood that the guidance placement system 10 can incorporate or be used with any appropriate preoperatively or intraoperatively acquired image data.

The guidance placement system 10 includes an imaging device 12 that is used to acquire pre-, intra-, or post-operative or real-time image data of the patient 16. Alternatively, various imageless system can be used. It will be understood, however, that patient image data are generally acquired using the imaging device 12 such as a point-of-care CT scanner discussed above and herein. As illustrated in FIG. 1, according to an exemplary form of the present disclosure, a patient 16 is scanned with the CT scanner 12 which include an x-ray source 11 and x-ray detector 13 revolving around a body of the patient 16 such as a head 18. In addition, the CT scanner 12 further includes a control unit 14 generating and storing a 3D image which is forwarded to the user interface unit 20. It will also be understood that the image data may be directly transmitted to the user interface unit 20, and also the control unit 14 of the CT scanner 12 can generate a 2D or 4D image if needed.

As illustrated in FIG. 1, the user interface unit 20 includes a display 22 showing images scanned and transmitted from the CT scanner 12, an input device 24 enabling a user to interface with the user interface unit 20, such as a touchpad, touch pen, touch screen, keyboard, mouse, or a combination thereof, and a control module 26 computing a planned trajectory for the guidance placement system 10. In addition, a planning tool software is installed in the control module 26 of the user interface unit 20 for processing the scanned images and generating an implementation plan or a surgical plan having prescribed parameters for guiding the placement of the medical instrument 28 such as the EVD catheter, exemplary discussed herein, in the head 18 of the patient 16.

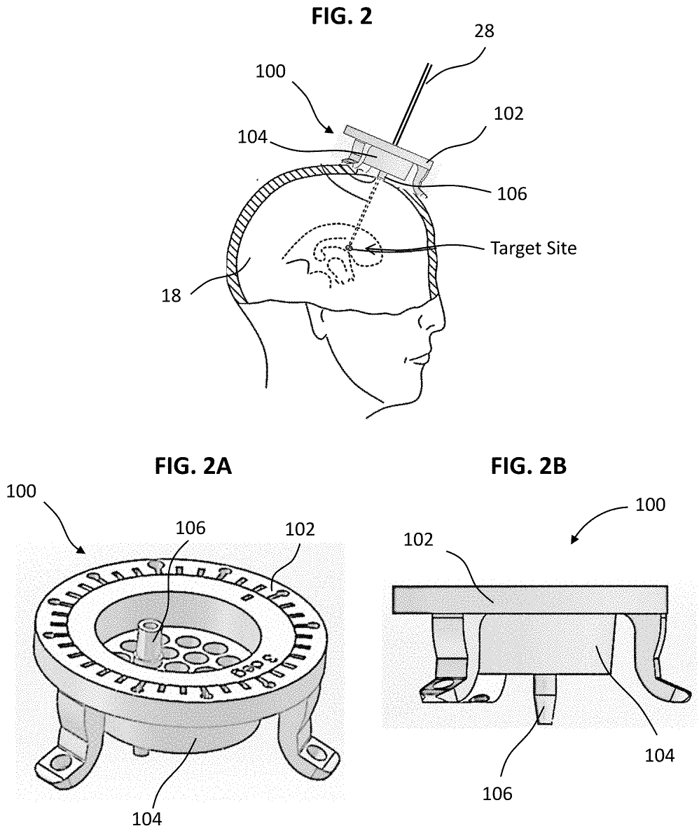

Also, as illustrated in FIG. 1, the guidance placement system 10 further includes an instrument placement kit 100 having an alignment frame 102, multiple guidance devices 104, and multiple sleeves 106 for guiding the placement of the medical instrument 28 in a body of the patient 16. In the guidance placement system 10, the instrument placement kit 100 is utilized for guiding the placement of the medical instrument 28 in the patient 16 according to the implementation plan generated from the control module 14 of the user interface unit 20. As illustrated in FIGS. 1 and 2, for example, the alignment frame 102 is present on the head 18 of the patient 16 during scanning the head 18 of the patient 16 using the CT scanner. The guidance devices 104 and the sleeves 106 are each configured and engaged with the alignment frame 102 (see FIGS. 2-2B) based on the prescribed data generated from the control module 26 having the planning software to support achieving the surgical plan. Accordingly, the guidance placement system 10 achieves a planned trajectory using surgical instrument such as an EVD catheter, biopsy needle, or other surgical instrumentation.

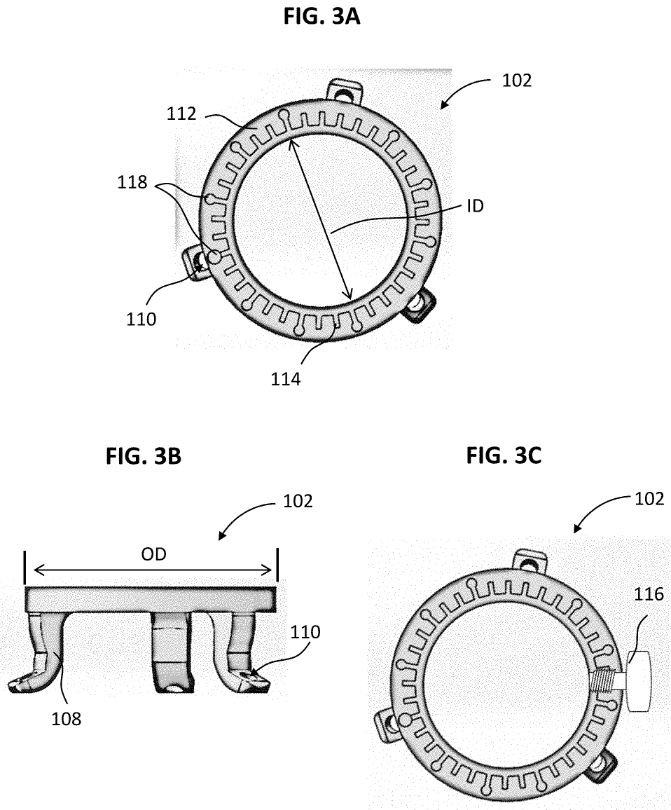

FIGS, 3A and 3B illustrate an exemplary embodiment of the alignment frame 102 formed as a ring shape such as, for example, a mini stereotactic frame. The alignment frame 102 is configured as a ring to be attached to a head 18 of the patient 16 adjacent an entry point of the head 18 for guiding the placement of the medical instrument 28 (see FIGS. 1 and 2). The attached alignment frame 102 serves as a base of the guidance devices 104 and also facilitates co-registered image-guided minimally-invasive intracranial procedures such as ventricular shunt placement or needle biopsy. As shown in FIG. 3B, the alignment frame 102 is formed with legs 108 to support the annular or ring shaped frame 102 in a stable manner and is securely attached to the body of the patient 16. As shown in an example of FIG. 3B, due to three legs 108 forming a tripod design, the alignment frame 102 avoids any rocking or instability associated with other configurations of legs. In addition, an end of each leg 108 is formed with a hole 110 for affixing to the body of the patient 16 via an adhesive, small bone screws, sutures, staples, or wires. In accordance with other forms of the present disclosure, however, the alignment frame may be formed with other design shapes with other attachment features to securely serve as the base, and also more legs or less legs for stability may be formed as needed.

As shown in FIGS. 3A and 3B, the alignment frame 102 has a bore or interior space defining an inner diameter ID, which has enough clearance for engaging with the guidance devices 104. For example, the inner diameter ID and the outer diameter OD of the alignment frame 102 are each generally between 50 mm˜150 mm. Preferably, the outer diameter OD of the alignment frame 102 is around 120 mm, and the inner diameter ID of the alignment frame 102 is around 110 mm, which may be enough to create skin incision (around 20˜30 mm) and a burr hole (around 15 mm) with tolerance to accommodate 10˜20 mm of potential alternative entry positions if needed. In addition, the alignment frame 102 is sufficiently rigid to retain its shape during at least single use and initial sterilization. For example, the alignment frame 102 may be formed by the 3D print with medical grade Nylon/Polyamide materials using SLS (selective laser sintering) additive manufacturing process. In accordance with other forms of the present disclosure, however, the alignment frame may be formed by other manufacturing processes or methods with other medical grade materials.

Furthermore, the alignment frame 102 includes a top surface 112 having markings 114 around the perimeter of the frame 102 to indicate positioning of the guidance devices 104 when one of the guidance devices 104 is engaged with the alignment frame 102. The frame 102 and device 104 may have corresponding flanges or shoulders sized and configured to allow the device to rest within the interior space of the frame 102. The alignment frame 102 further includes a locking feature 116 such as a thumbscrew or wingnut bolt for removably attaching one of the guidance devices 104 and/or other instrument guides for making incision, location of burr hole, or other procedural steps. As shown in FIG. 3C, at least one thumbscrew 116 would come in through the side of the alignment frame 102 and bind on the guidance device 104 such that more thumbscrews 116 may be used for securing the guidance device 104. The thumbscrew 116 is also made of nylon in order not to interfere with the scanned images taken by the CT scanner and is easily tightened by the user to secure the guidance device 104 when the selected guidance device is in engagement with the alignment frame 102. Furthermore, the locking feature 116 supports the stable engagement between the alignment frame 102 and the guidance devices 104 during drilling for making an entry hole of a burr hole on the body of the patient 16. The skilled artisan will recognize that various mechanical or electromechanical locking devices may be used, including slots receiving projections, tabs and detects, frictional engagements, and actuated locks like a deadbolt.

In addition, as shown in FIG. 3A, the alignment frame 102 includes one or more identifiers such as radio-opaque fiducial markers 118 which are highly visible on the CT scanner including a low-dose CT scanner, which can be used to accurately locate the frame structure relative to the patent bony anatomical landmarks including a target site inside the body of the patient 16. In the scanned images from the CT scanner, the fiducial markers 118 of the alignment frame 102 is used as a reference plane for determining the planned trajectory of the surgical procedure. In FIG. 3A, for example, ten (10) radio-opaqued fiducial markers 118 with a circular or dot shape are embedded in or printed on the top surface 112 around the perimeter of the alignment frame 102. The markers 118 may take any shape, but preferably are spheres or discs embedded into the frame 102. One of the pluralities of fiducial markers 118 has a bigger size than other fiducial markers 118 to indicate the north direction which means 0 degree of the ring angle as a pre-defined parameter. Accordingly, when one of the guidance devices 104 is engaged with the alignment frame 102, the rotational position of the guidance device 104 is measured from the north direction fiducial marker indicated by one of the fiducial markers 118 having the bigger size of the dot. In addition, when each of the guidance devices 104 is engaged with the alignment frame 102, the guidance devices 104 are rotated or spined in a discrete manner or a continuous manner.

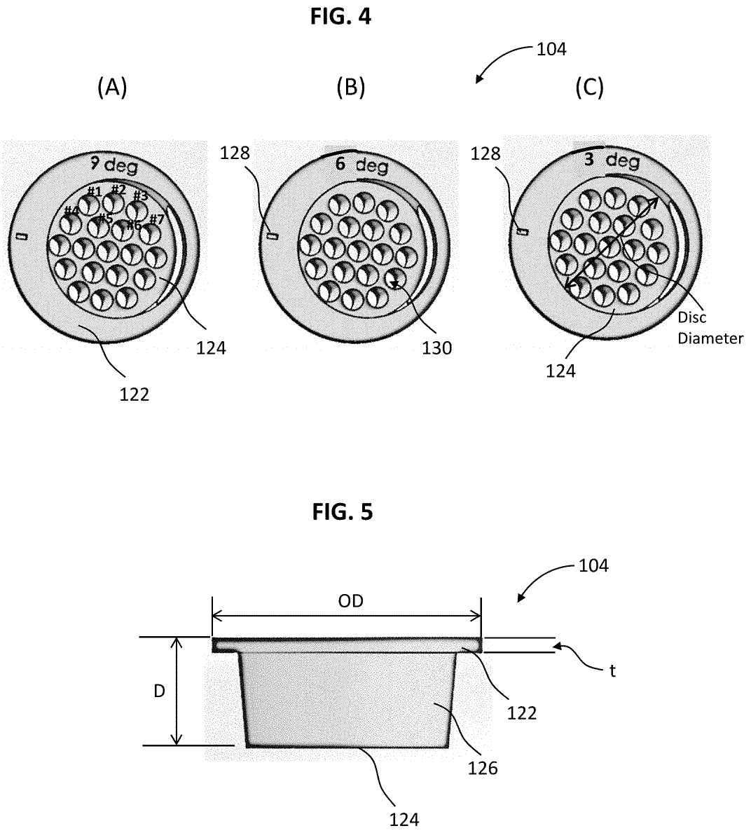

Referring to FIGS. 4(A), 4(B), 4(C), and 5, the instrument placement kit 100 further includes the multiple guidance devices 104 each configured for selective engagement with the alignment frame 102 as shown in FIG. 2A. Each guidance device 104 has a set of pre-defined parameters such as a given number of holes (each having a hole number) and a hole angle (an inclination of the bore forming the hole relative to the upper surface of the guidance device 104). As shown in FIGS. 4(A)-4(C), each hole number of the multiple holes 130 indicates an insertion location (a placement location) of the medical instrument 28 as an entry point of the medical instrument 28 and a common hole angle of each guidance device 104 indicates an insertion angle of the medical instrument for guiding its placement. In FIGS. 4(A)-4(C), and 5, each of the guidance devices 104 is formed as a circular shape having an upper flange 122 and a bottom disc 124 extending from a plane of the upper flange 122, such that the upper flange 122 and the bottom disc 124 are parallel and connected to each other by a circular side wall 126. The upper flange 122 of the guidance device 104 has a point marker 128, which is marked on the top surface of the upper flange 122 such that the guidance device 104 enables a user to indicate an insertion direction of the medical instrument 28 (for example, a rotational position of the guidance device). The point marker 128 of the guidance device 104 indicate desired orientation relative to the base alignment frame 102. Accordingly, as shown in FIG. 2A, the point marker 128 of the guidance device 104 aligns with one of the markings 114 of the alignment frame 102 according to one (the ring angle) of the prescribed parameters determined from the control module 26 of the user interface unit 20 when one of the guidance devices 104 is engaged with the alignment frame 102. When the selected guidance device 104 is in engagement with the alignment frame 102, furthermore, the top surface of the upper flange 122 and the top surface 112 of the alignment frame 102 stay flush such that they are in a co-planar,

As shown in FIGS. 4(A)-4(C), the guidance devices 104 are each formed with multiple holes 130 arranged as a honeycomb pattern on the bottom disc 124 to indicate an insertion location (a placement location) of the medical instrument 28 according to one (the hole number) of the prescribed parameters. However, in accordance with other forms of the present disclosure, the multiple holes may be formed with other patterns such as an in-line pattern or a rectangular pattern. Each hole 130 of the guidance device 104 is numbered according to the pre-defined pattern, and one hole's number of the guidance device 104 is displayed as one of the prescribed parameters in the user interface unit 20.

In addition, all of the holes 130 formed through the bottom disc 124 of each guidance device 104 are parallel to each other and are at a same angle, which is inclined relative to the top surface of the bottom disc 124 to indicate an insertion angle (an inclined angle) of the medical instrument 28. In other embodiments, each hole may be at a different angle, and/or they may be inclined relative to a common point such as the center of the guidance device.) For example, the specific angle of each guidance device 104 is relative to a vertical line Z, which is normal to the surface of the bottom disc 124 (see FIGS. 4(A)-4(C), and 5. In the guidance device 104, for example, “0 degree” mark on the surface of the bottom disc 124 means that all of the holes 130 formed in the bottom disc 124 are perpendicular to the surface of the bottom disc 124 such that all holes' angle is 0 degree. Furthermore, the hole angulation of the guidance device 104 goes “up” on the disc 124 (towards an opposite direction from the point marker 128 formed on the upper flange 122) in order to correlate with the user directional intuition.

According to an exemplary form of the present disclosure, the instrument placement kit 100 generally includes a set of six (6) guidance devices 104 each having a specific hole angle such as 0/3/6/9/12/15 degree, which is printed on the top surface of the guidance device 104 such that the user can easily select one of the guidance devices 104 according to the prescribed hole angle from the user interface unit 20. As an example, FIGS. 4(A)-4(C) show three (3) guidance devices having three different hole angles such as 3 degrees, 6 degrees, and 9 degrees. In accordance with other forms of the present disclosure, however, the specific hole angle on each of the guidance devices 104 may be indicated with the color-coded method instead of marking the hole angle on the upper surface. In addition, the kit 100 may have more guidance devices 104 with more specific hole angles such that the number of the guidance devices 104 having the specific hole angles may be varied. For example, the instrument placement kit 100 includes a set of ten (10) guidance devices 104 each having their specific hole angle as discussed above such that the user can select one of the ten different hole angles.

As shown in FIGS. 4(A)-4(C), and 5, the outer diameter OD of the upper flange 122 of the guidance device 104 is generally between 50 mm˜150 mm,and preferably around 110 mm. The diameter of the bottom disc 124 is generally between 25 mm˜125 mm, and preferably around 55 mm. The depth D of the bottom disc 124 from top surface of the upper flange 122 generally between 25 mm˜30 mm, and preferably around 22 mm, and the thickness t of the upper flange 122 is generally between 1 mm˜5 mm, and preferably around 3 mm. In addition, the diameter of each hole 130 is generally 2 mm˜10 mm, and preferably around 6 mm. The size of the holes 130 formed on the bottom disc 124 is large enough to support for a skull drill to drill an entry hole of the medical instrument 28 or a burr hole after one of the guidance devices 104 is engaged with the alignment frame 102 according to the prescribed parameters determined from the control module 26 of the user interface unit 20. For example, when the skull drill is operated after one of the guidance devices 104 is engaged with the alignment frame 102, if needed, a removable thin metal insert can be used to prevent debris during its drilling. Also, the skull drill would have a smooth shaft and only have the cutting blades at its end.

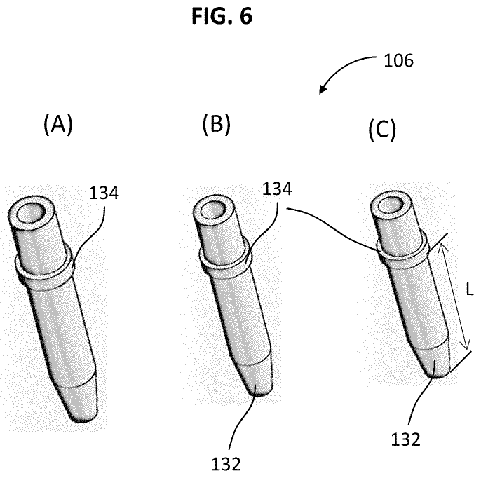

The instrument placement kit 100 further include multiple sleeves 106 each having different pre-defined lengths for guiding the placement of the medical instrument 28 such that the user interface unit 20 is also operable to determine a prescribed length among the different pre-defined lengths based on the planned trajectory. The sleeves 106 narrow the diameter of the selected hole 130 and can have presecribed inner diameters, e.g. to correspond to standard size catheters or other placement or guidance devices. As shown in FIGS. 6(A)-6(C), generally, the instrument placement kit 100 includes three or four (3 or 4) sleeves 106 each having different lengths, which are separated by 2 or 3 mm. In addition, each of the sleeves 106 is formed with a radial ring 134 to place on the top surface of the bottom disc 124 when each of the sleeves 106 is inserted into the selected hole 130 of the selected guidance device 104. Accordingly, the radial ring 134 of the sleeve 106 is configured to perform as a stopper. In addition, the pre-defined length L of the sleeve 106 is measured as a distance between the bottom surface of the radial ring 134 and a chamfered end 132. For example, FIGS. 6(A)-6(C) illustrate the sleeves 106 each having a specific length, which is printed on a surface of the sleeve 106 for the user to recognize the pre-defined length of each sleeve 106. In accordance with other forms of the present disclosure, however, the length of the sleeves 106 may be recognized based on the different color-code of the length.

The user interface unit 20 is operable to select one of the sleeves 106 based on the prescribed length, and the selected sleeve 106 is engaged with the selected guidance device 104, which is mounted to the alignment frame 102. As shown in FIG. 2A, the selected sleeve 106 is inserted into the selected hole 130 as the entry point of the medical instrument 28 in the selected guidance device 104. As described above, the selected hole 130 of the guidance device 104 for drilling the entry hole with the skull drill as the entry point of the medical instrument 28 is also used for inserting the selected sleeve 106 for guiding the placement of the medical instrument 28. Due to the sleeve 106 inserted into the hole 130 of the guidance device 104, the diameter of the hole 130 is decreased by 2˜3 mm such that the hole diameter of the inserted sleeve 106 for the EVD catheter insertion becomes around 3˜4 mm. The inserted sleeve 106 is securely engaged with the selected hole 130 and allows the user to achieve the guided placement of the medical instrument 28 (for example, the EVD catheter or biopsy needle) based on the planned trajectory determined by the user interface unit 12. In addition, each of the sleeves 106 is formed with the chamfered end 132, which contacts and is engaged with the entry hole on the body of the patient 16 for stability when the medical instrument 28 inserted into the body of the patient 16 based on the planned trajectory.

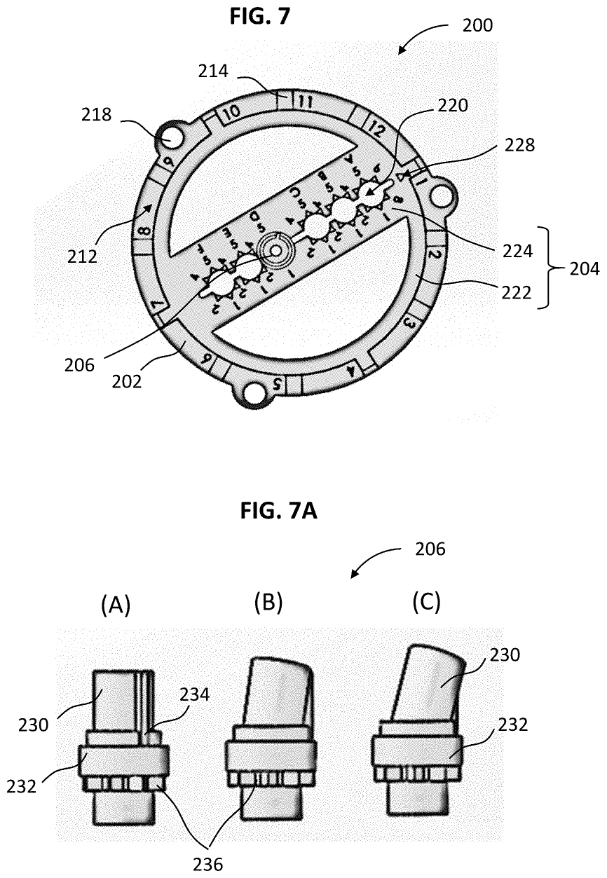

FIG. 7 illustrates a modified instrument placement kit 200 as the second embodiment of the present disclosure. In the second embodiment of the present disclosure, the instrument placement kit 200 includes an alignment frame 202, a guidance device 204, and multiple guide carriages 206, which are generally similar to each component of the first embodiment (see FIG. 1B). As shown in FIG. the alignment frame 202 is generally same as the alignment ring frame 102 in the first embodiment other than the pattern of markings 214 and identifiers 218 (fiducial markers) formed on the top surface 212 of the alignment frame 202. The markings 214 indicate a rotational position of the guidance devices 204 when one of the guidance devices 204 is rotatably engaged with the alignment frame 202.

The guidance device 204 in the second embodiment of the present disclosure is formed as a circular shape having an outer ring 222 and a middle rail 224 formed in a single plane. The middle rail 224 is connected to the outer ing 222 along a center line of the outer ring 222 and multiple holes 220 arranged in a line. The outer ring 222 is formed with a point marker 228, which is marked on the top surface of the outer ring 222 and aligns with one of the markings 214 of the alignment frame 202. Accordingly, the guidance device 204 with the multiple holes 220 and the point maker 228 in the second embodiment is configured to determine an insertion location of the medical instrument based on the prescribed parameter determined from the planned trajectory.

In the second embodiment of the present disclosure, the instrument placement kit 200 further includes multiple guide carriages 206 each having different sets of pre-defined parameters. As shown in FIGS. 7 and 7A, one of the multiple guide carriages 206 is inserted into one of the multiple holes 220 of the guidance device 204 to guide the placement of the medical instrument according to the prescribed parameters determined from the planned trajectory, which is computed in the same way as in the first embodiment. Each of the multiple guide carriages 206 is formed with a sleeve 230 and a radial ring 232, and removably engaged with one of the holes 220.

As shown in FIGS. 7 and 7A, the guide carriage 206 further includes a pointer 234 for indicating the rotational position of the carriage 206 when the selected carriage 206 is engaged with one of the holes 220, which indicates an insertion direction of the medical instrument 28 as one of the prescribed parameters. Also, the radial ring 232 is formed with a keying feature 236 such as a star shape on the bottom surface of the radial ring 232 to securly lock the guide carriage 206 into the middle rail 224 with exact orientation. For engaging with the key feature 236 of the guide carriage 206, a mating feature around each hole 220 of the middle rail 224 are formed as shown in FIG. 7. In addition, each of the carriages 206 is formed with an angle (a hole angle) which is inclined relative to a longitudinal axis of the sleeve 230. The inclined angle inside the sleeve 230 indicates an insertion angle of the medical instrument 28 as one of the prescribed parameters. Accordingly, one of the multiple guide carriages 206 is selected and engaged with one of the holes 220 formed on the middle rail 224 of the guidance device 204 as shown in FIG. 7. The user interface unit 20 is operable to generate the prescribed parameters of the instrument placement kit 200 in the second embodiment, which is the same way as the first embodiment of the present disclosure described above.

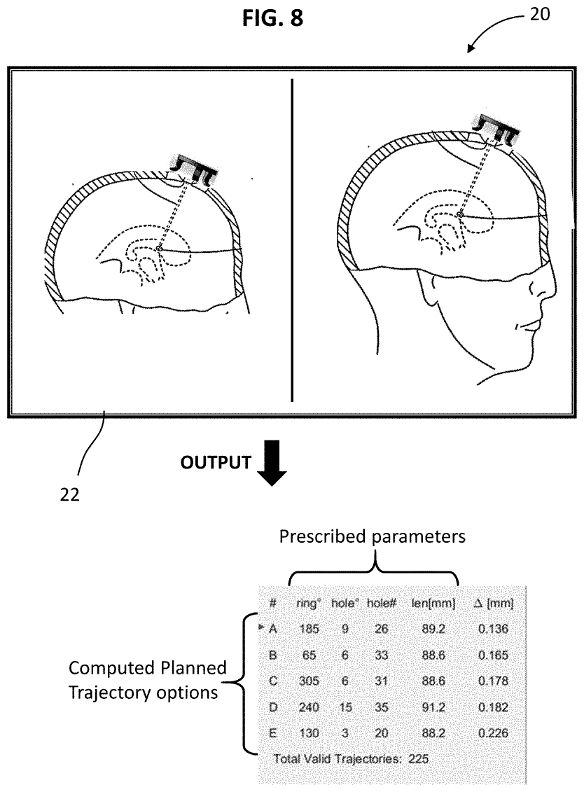

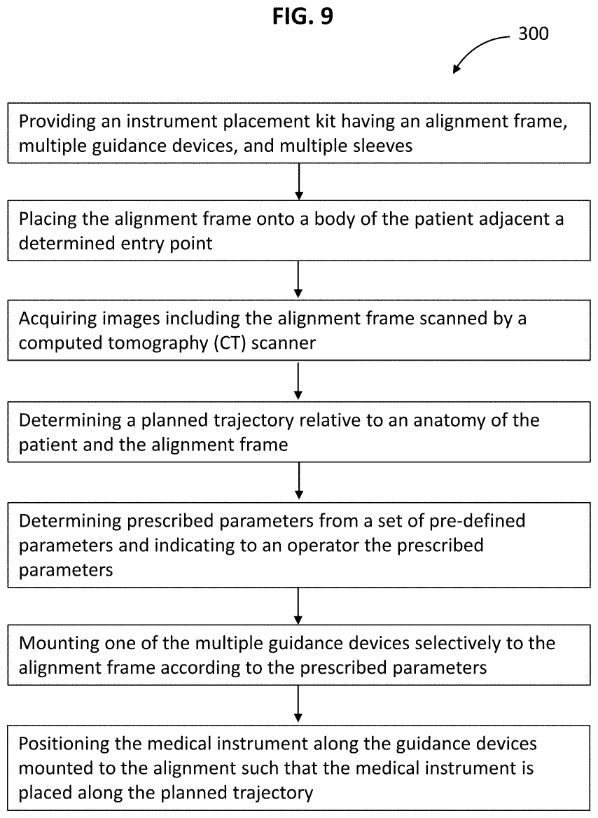

FIG. 8 shows a display screen showing the scanned images and the prescribed parameters as an output in the user interface unit 20 of the guidance placement system 10, and FIG. 9 shows a flow diagram 300 of one method for the guided placement of a medical instrument 28 in a patient using the guided placement system 10 of the present disclosure as described above. In accordance with an exemplary form of the present disclosure, the instrument placement kit 100 having the alignment frame 102, multiple guidance devices 104, and multiple sleeves 106 is provided. The alignment frame 102 is placed or affixed onto the body of the patient 16 adjacent a determined entry point. For example, as shown in FIGS. 1 and 2, when the alignment frame 102 is attached to the head 18 of the patient 16, the alignment ring frame 102 is placed onto the patient's head centered on Kocher's point defined by “Three Knuckle” rule of thumb. After placing the alignment ring frame 102 onto the patient's head, the images including the brain of the patient with the alignment ring frame 102 having the radio-opaqued fiducial markers 118 scanned by the CT scanner 12 are acquired and transmitted to the user interface unit 12. which is shown in FIGS. 1 and 8.

In FIG. 8, the control module 26 of the user interface unit 12 has an installed planning software to calculate an ideal medical instrument or catheter insertion trajectory based on the scanned images. As shown in FIG. 8, the user interface unit 12 displays the acquired scanned images having the patient anatomical structures such as the skull and ventricles, and a reference plane defined by the fiducial markers 118 of the alignment frame 102. The control module 26 may overlay a pre-procedure imaging (e.g., MRI or CT-V) to provide other ancillary information such as location of peripheral veins, neural tractography, suspected tumor tissue or lesion. Also, the control module 26 can be automated by computer algorithms such as machine learning based method or other methods for calculating optimal and safe insertion pathways for guiding the placement of the medical instrument. The set of optimal, safe paths can be discretized to conform to the discrete set of positions and angles easily achievable using the Alignment Ring Frame and Guidance devices. The surgical plan is also manually by a physician or medical technician in a multiplanar view by clicking on the entry point of the medical instrument and clicking on the target site to define a trajectory. Based on the planned trajectory, the control module 26 outputs the prescription specifying the configuration of the guidance devices 104 relative to the alignment frame 102 and the sleeves 106 such that the control module 26 determines the prescribed parameters from a set of pre-defined parameters. The system prescribes allows an operator to select the prescribed parameters such as a particular guidance device 104 among the set of guidance devices, a ring angle which indicates a rotational position of the guidance device 104 (e.g., an insertion direction of the medical instrument) relative to the alignment frame 102, a hole angle which indicates an inclined angle of the multiple holes 130 formed on the guidance device 104 (e.g., an insertion angle of the medical instrument), a hole number which indicates a placement location of the medical instrument 28 (e.g., an insertion location of the medical instrument), and a sleeve size and/or length which indicates a distance between the entry point of the patient body and the bottom disc 124 (e.g., an insertion length of the medical instrument). As shown in FIG. 8, generally, the control module 26 provides multiple planned trajectory options each having its specific prescribed parameters. In addition, a vein map inside the patient's body can be optionally provided with the prescribed parameters.

As described in the diagram 300 of FIG. 9, one of the guidance devices 104 is selected and mounted to the alignment frame 102 according to the prescribed parameters generated from the control module 26. FIG. 8 shows the multiple outputs of the prescribed parameters based on the planned trajectory for guiding the placement of the medical instrument 28. One of the guidance devices 104 is selected according to the prescribed hole angle (which is the inclined angle of the multiple holes 130) and rotatably positioned in the alignment frame 102 according to the ring angle (which is the rotational position of the guidance device 104). In the selected guidance device 104, one of the multiple holes 130 is also selected according to the hole number (which is the placement location of the medical instrument 28). As shown in FIGS. 2 and 2A, accordingly, one of the guidance devices 104 is selected based on the prescribed parameters and the selected guidance device 104 is engaged with the alignment frame 102.

As shown in FIG. 9, furthermore, an entry hole on the body of the patient 16 is drilled as the entry point of the medical instrument 28 or a burr hole. For example, the entry hole on the skull of the patient 16 is drilled with a skull drill through the selected hole 130 of the guidance devices 104 mounted to the alignment frame 102. In addition, the multiple sleeves 106 are provided with different pre-defined lengths for guiding an insertion of the medical instrument 28 through the entry hole drilled on the skull of the patient 16. One of the multiple sleeves 106 is selected according to a prescribed length determined from the planned trajectory. The selected sleeve 106 is inserted into the selected hole 130 formed on the bottom disc 124 of the guidance device 104, which was previously used for the skull drill to make the entry hole on the skull of the patient as described above. In addition, the chamfered end 132 of the selected sleeve 106 according to the prescribed length is engaged with the entry hole for stability when the medical instrument 28 is inserted into the target site of the patient body through the engaged sleeve 106, Accordingly, the medical instrument 28 such as the EVD catheter or biopsy needle is effectively placed in the body of the patient along the planned trajectory generated from the scanned images as described above.

Each of the above described elements may be used with the method described above or other methods. Further, each of the described elements may be used together or independently. Further, each alternative for one element may be utilized in combination with each alternative for other elements.

The methods, elements, processing, and logic described above may be implemented in many different ways and in many different combinations of hardware and software. For example, all or parts of certain elements may be performed with circuitry that includes an instruction processor, such as a Central Processing Unit (CPU), microcontroller, or a microprocessor; an Application Specific Integrated Circuit (ASIC), Programmable Logic Device (PLD), or Field Programmable Gate Array (FPGA); or circuitry that includes discrete logic or other circuit components, including analog circuit components, digital circuit components or both; or any combination thereof. The circuitry may include discrete interconnected hardware components and/or may be combined on a single integrated circuit die, distributed among multiple integrated circuit dies, or implemented in a Multiple Chip Module (MCM) of multiple integrated circuit dies in a common package, as examples.

The circuitry may further include or access instructions for execution by the circuitry. The instructions may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other optical disk; or in or on another machine-readable medium. A product, such as a computer program product, may include a storage medium and instructions stored in or on the medium, and the instructions when executed by the circuitry in a device may cause the device to implement any of the processing described above or illustrated in the drawings.

The implementations may be distributed as circuitry among multiple system components, such as among multiple processors and memories, optionally including multiple distributed processing systems. Parameters, databases, and other data structures may be separately stored and managed, may be incorporated into a single memory or database, may be logically and physically organized in many different ways, and may be implemented in many different ways, including as data structures such as linked lists, hash tables, arrays, records, objects, or implicit storage mechanisms. Programs may be parts (e.g., subroutines) of a single program, separate programs, distributed across several memories and processors, or implemented in many different ways, such as in a library, such as a shared library (e.g., a Dynamic Link library (DLL)). The DLL, for example, may store instructions that perform any of the processing described above or illustrated in the drawings, when executed by the circuitry.

The foregoing description of various forms of the invention has been presented for purposes of illustration and description. It is not intended to be exhaustive or to limit the invention to the precise forms disclosed. Numerous modifications or variations are possible in light of the above teachings. The forms discussed were chosen and described to provide the best illustration of the principles of the invention and its practical application to thereby enable one of ordinary skill in the art to utilize the invention in various forms and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A system for a guided placement of a medical instrument in a patient with a computed tomography (CT) scanner providing scanned images of the patient including a target site for the medical instrument, the system comprising:
an instrument placement kit including,
an alignment frame configured to be attached to a body of the patient, the alignment frame having one or more identifiers to define a reference plane that can be determined from scanned images from the CT scanner; and
one or more guidance devices each configured for selective engagement with the alignment frame, each guidance device having a set of pre-defined parameters, the one or more guidance devices having different sets of the pre-defined parameters for guiding the placement of the medical instrument; and
a user interface unit operable to determine the pre-defined parameters of the one or more guidance devices from the scanned images including the reference plane of the alignment frame,
wherein the user interface unit is operable to compute a planned trajectory of the medical instrument based on the target site and the reference plane of the alignment frame, the user interface unit is further operable to determine prescribed parameters among the different sets of pre-defined parameters and configure a guidance device from the one or more guidance devices, and the user interface unit is operable to indicate the prescribed parameters such that the one or more guidance devices can be configured and selectively mounted to the alignment frame according to the prescribed parameters to guide the medical instrument along the planned trajectory.

2. The system of claim 1, wherein the alignment frame is formed as a ring shape with markings around a perimeter of the alignment frame for indicating a rotational position of the selected guidance device when the selected guidance device is engaged with the alignment frame.

3. The system of claim 1, wherein each of the guidance devices is formed as a circular shape having an upper flange and a bottom disc extending from a plane of the upper flange such that each of the guidance devices is formed with the upper flange and the bottom disc, which are parallel and connected with a circular side wall.

4. The system of claim 3, wherein the upper flange of the guidance device has a marker to align with one of markings formed around a perimeter of the alignment frame to indicate an insertion direction of the medical instrument as one of the pre-defined parameters.

5. The system of claim 3, wherein the bottom disc of the guidance device is formed with multiple holes spaced in a regular pattern or irregular pattern for indicating an insertion location of the medical instrument as one of the pre-defined parameters and all of the holes formed through the bottom disc of the guidance device are parallel to each other and are at a same angle, which is inclined relative to a top surface of the bottom disc for indicating an insertion angle of the medical instrument as one of the pre-defined parameters.

6. The system of claim 1, wherein the instrument placement kit further includes multiple sleeves each having different pre-defined lengths for guiding the placement of the medical instrument such that the user interface unit is operable to determine a prescribed length among the different pre-defined lengths based on the planned trajectory.

7. The system of claim 6, wherein the user interface unit is further operable to select a sleeve from the multiple sleeves based on the prescribed length such that the selected sleeve can be selectively engaged with the selected guidance device mounted to the alignment frame.

8. The system of claim 7, wherein the selected sleeve is inserted into one of multiple holes formed through a bottom disc of the selected guidance device determined as an entry location of the medical instrument.

9. The system of claim 8, wherein each of the sleeves has an end for engaging with an entry hole drilled on the body of the patient when the medical instrument is placed in the body of the patient along the planned trajectory.

10. The system of claim 1, wherein each of the guidance devices is formed as a circular shape having an outer ring and a middle rail formed along a center line of the outer ring such that the outer ring and the middle rail are formed in a single plane.

11. The system of claim 10, wherein the outer ring of the guidance device is formed with a marker to align with one of markings formed around a perimeter of the alignment frame and the middle rail of the guidance device is formed with multiple holes, and wherein the aligned marker and selected hole of the guidance device indicate an insertion location of the medical instrument as one of the pre-defined parameters.

12. The system of claim 11, wherein the instrument placement kit further includes one or more guide carriages each having different sets of pre-defined parameters such as an insertion angle and an insertion direction of the medical instrument for guiding the placement of the medical instrument such that the user interface unit is operable to select one among the guide carriages and also specify the configuration and orientation of the carriage based on the prescribed parameters determined from the planned trajectory.

13. A method for a guided placement of a medical instrument in a patient, the method comprising the steps of:
providing an instrument placement kit;
placing an alignment frame onto a body of the patient adjacent a determined entry point;
acquiring images including the alignment frame scanned by a computed tomography (CT) scanner;
determining a planned trajectory relative to an anatomy of the patient and the alignment frame;
determining prescribed parameters from a set of pre-defined parameters and indicating to an operator the prescribed parameters;
mounting one of multiple guidance devices selectively to the alignment frame according to the prescribed parameters; and
positioning the medical instrument along the guidance devices mounted to the alignment frame such that the medical instrument is placed along the planned trajectory.

14. The method of claim 13, wherein the step of determining the planned trajectory relative to the anatomy of the patient and the alignment frame includes the step of indicating a target site in the acquired scanned images having the anatomy of the patient and a reference plane derived from identifiers embedded in the alignment frame.

15. The method of claim 13, wherein the step of mounting one of the multiple guidance devices selectively to the alignment frame includes the steps:
selecting one of the guidance devices each having one inclined angle of multiple holes formed in each of the guidance devices according to a prescribed hole angle; and
rotatably positioning the selected guidance device in the alignment frame according to a prescribed ring angle.

16. The method of claim 13, wherein the step of positioning the medical instrument along the guidance devices mounted to the alignment frame includes the steps of:
selecting one of multiple holes formed in each of the guidance devices as an insertion location of the medical instrument according to a prescribed hole number; and
making an entry hole on the body of the patient at the insertion location of the medical instrument through the selected hole of the selected guidance device engaged with the alignment frame.

17. The method of claim 16 further comprising the step of providing one or more sleeves each having different pre-defined lengths for guiding an insertion length of the medical instrument through the entry hole on the body of the patient.

18. The method of claim 17, wherein the step of providing the one or more sleeves includes the steps of selecting one sleeve among the sleeves having the different pre-defined lengths according to a prescribed length determined from the planned trajectory; inserting the selected sleeve into the selected hole of the selected guidance device; and engaging an end formed in each of the sleeves with the entry hole on the body of the patient.

19. A guided placement device for a medical instrument in a patient along a planned trajectory determined from a user interface unit having a control module, the guided placement device comprising:
an alignment frame attached to a body of the patient, the alignment frame having multiple markings to indicate an insertion direction of the medical instrument; and
a guidance device selectively engaged with the alignment frame in one of multiple discrete rotational positions, upper surfaces of the guidance device and alignment frame being coplanar when engaged, the guidance device having a marker to align with one of the markings in a discrete rotational manner, the guidance device having multiple holes representing different insertion locations of the medical instrument.

20. The guided placement device of claim 19, wherein the device further includes multiple sleeves selectively engaged with one of the holes arranged in the selected guidance device mounted to the alignment frame to guide an insertion length of the medical instrument.

21. The guided placement device of claim 19, wherein all of the holes formed through a plane of the guidance device are parallel to each other and are at a same angle, which is inclined relative to a surface of the plane for indicating an insertion angle of the medical instrument.

22. The guided placement device of claim 19, wherein the holes formed in the guidance device are arranged in a honeycomb pattern.

23. The guided placement device of claim 19, wherein the alignment frame includes a number of radio-opaque fiducial markers arranged in a regular pattern to define a reference frame of the device.
